(19) United States
(12) Patent Application Publication
(10) Pub. No.: US 2018/0206796 A1
(43) Pub. Date: Jul. 26, 2018

(54) SYSTEM FOR PRESENTING CHANGES IN PULSES BY MEANS OF PULSE ANALYSIS APPLICATION

(71) Applicant: Bong Jae JEON, Hwaseong-si (KR)

(72) Inventor: Bong Jae JEON, Hwaseong-si (KR)

(21) Appl. No.: 15/861,941

(22) Filed: Jan. 4, 2018

(30) Foreign Application Priority Data
Jan. 23, 2017 (KR) 10-2017-0010369

(51) Int. Cl. A61B 5/00; A61B 5/024

(52) U.S. Cl. CPC A61B 5/743; A61B 5/0004; A61B 5/002; A61B 5/7415; A61B 5/6898; A61B 5/7282; A61B 5/02438

(57) ABSTRACT

The present invention relates to a system for presenting changes in pulses by means of a pulse analysis application, which detects a user's own pulse and the pulse of another person, calculates synchronization between changes in the user's own pulse and the pulse of the other person by means of a pulse analysis application of a smartphone, and presents the result of the calculation, thereby enabling the climax of a M mutual change in pulses to be determined. The system includes: a plurality of biological signal sensors configured to acquire biological signals, and to wirelessly transmit the acquired biological signals; and a biological information checking terminal configured to display the biological signals transmitted by the plurality of biological signal sensors, to calculate synchronization between the plurality of biological signals by means of a pulse analysis application, and to display a result of the calculation as an event.

### BACKGROUND

### 1. Technical Field

The present invention relates generally to a system for presenting changes in pulses by means of a pulse analysis application, and more specifically to a system for presenting changes in pulses by means of a pulse analysis application, which detects a user's own pulse and the pulse of another person, calculates synchronization between changes in the user's own pulse and the pulse of the other person by means of a pulse analysis application of a smartphone, and presents the result of the calculation, thereby enabling the climax of a mutual change in pulses to be determined.

### 2. Description of the Related Art

With the diversification of the functions of smartphones, there has been known a technology for detecting a pulse, analyzing the detected pulse by means of a pulse analysis application, and/or representing the result of the analysis.

In particular, in the case of the Xiaomi Mi Band, it is simply worn on a wrist, and detects a pulse by means of a smartphone.

Furthermore, conventional technologies for detecting biological information by means of an information device, such as a smartphone, and presenting the biological information, thereby enabling health to be managed or enabling a biologically excited state to be presented are disclosed in Korean Patent No. 10-1249274 (issued on Mar. 26, 2013) entitled “Biological Signal Self-diagnosis System using Smartphone (hereinafter referred to as “patent document 1”), Korean Patent Application Publication 10-2015-0039113 (published on Apr. 9, 2015) entitled “Method and Device for processing Content based on Biological Signal” (hereinafter referred to as “patent document 2”), and Korean Patent Application Publication 10-2016-0150347 (published on Dec. 30, 2016) entitled “Cardiovascular Assessment System and Program based on Interaction between Smart device and Wearable Device” (hereinafter referred to as “patent document 3”).

The conventional technology disclosed in “patent document 1” is directed to a system for receiving biological signals, measured by a portable health meter, over a short-distance communication network or the like, collecting the biological signals, measured by the portable health meter, through the continuous storage of them in a smartphone, and generating diagnosis data. Accordingly, the collected data can be used as a personal health diary. Furthermore, a previously measured biological signal and a currently measured biological signal can be compared with each other, and then an action guide, such as the volume of drink, the type of alcohol to be taken, etc., based on a personal health state can be proposed.

Furthermore, the conventional technology disclosed in “patent document 2” is directed to a method of processing content based on a biological signal. The conventional technology is configured to acquire a biological signal of a user, to determine the type of content to be output or a parameter adapted to change a characteristic based on the acquired biological signal, to process or replace content based on the determined parameter, and to output the processed or replaced content.

Moreover, the conventional technology disclosed in “patent document 3” is directed to a cardiovascular assessment system and program which are configured to measure volume pulses at different location of a human body by means of a smart device and a wearable device, to calculate a blood flow speed index and a blood pressure index based on the difference between the measured volume pulses, and to provide the calculated indexes as indexes for cardiovascular assessment.

### SUMMARY

However, although the above-described pulse detection and analysis method using a common smartphone and the above-described conventional technologies can detect and analyze a user's own pulse or the biological signal (in particular, the pulse) of a single person and display the result of the analysis, they are disadvantageous in that it is impossible to detect, analyze and display the pulse of another person and it is also impossible to calculate and present synchronization between a change in the pulse of another person and a change in the pulse of a user.

In other words, the conventional technologies are disadvantageous in that it is impossible to determine the pulse state of a counterpart in a specific state (in particular, in an excited state) in real time by means of an information device, such as a smartphone, and it is also impossible to calculate a change in the pulse of the counterpart and a change in the pulse of the user and then present the calculated changes in an integrated manner.

The present invention has been conceived to overcome the above-described problems, and an object of the present invention is to provide a system for presenting changes in pulses by means of a pulse analysis application, which detects a user's own pulse and the pulse of another person, calculates synchronization between changes in the user's own pulse and the pulse of the other person by means of a pulse analysis application of a smartphone, and presents the result of the calculation, thereby enabling the climax of a mutual change in pulses to be determined.

According to an aspect of the present invention, there is provided a system for presenting changes in pulses by means of a pulse analysis application, the system displaying synchronization between changes in pulses by means of a pulse analysis application of a smartphone, the system including: a plurality of biological signal sensors configured to acquire biological signals, and to wirelessly transmit the acquired biological signals; and a biological information checking terminal configured to display the biological signals transmitted by the plurality of biological signal sensors, to calculate synchronization between the plurality of biological signals by means of a pulse analysis application, and to display a result of the calculation as an event.

The plurality of biological signal sensors may be worn on the wrists of biological signal measurement target persons in band forms, may detect pulses as biological signals, may convert the detected biological signals into short-distance wireless signals, and may transmit the short-distance wireless signals.

The biological information checking terminal may further include: a biological signal reception unit configured to receive the biological signals transmitted by the plurality of biological signal sensors; and a display unit configured to visually display the received biological signals.

The biological information checking terminal may further include: a control unit configured to control analysis of a plurality of pieces of received biological information and to control an occurrence of an event based on a result of the analysis; and an application execution unit configured to execute the pulse analysis application in conjunction with the control unit.

The biological information checking terminal may further include a biological signal analysis unit configured to analyze the biological signals by means of a method of calculating synchronization between changes in first and second pulse signals by means of the pulse analysis application executed by the application execution unit.

The biological information checking terminal may further include an event generation unit configured to generate an event corresponding to the synchronization between the changes in pulses analyzed by the biological signal analysis unit under a control of the control unit.

The event generation unit may vary a sound effect or may automatically select and output corresponding music based on the result of the calculation of the synchronization between the changes in pulses.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a block diagram showing a system for presenting changes in pulses by means of a pulse analysis application according to a first embodiment of the present invention;

FIG. 2 is a block diagram showing a system for presenting changes in pulses by means of a pulse analysis application according to a second embodiment of the present invention; and

FIGS. 3a and 3b are diagrams showing examples of synchronization between changes in a user's own pulse and the pulse of another person according to the present invention.

### DETAILED DESCRIPTION

Systems for displaying a change in pulse using a pulse analysis application according to preferred embodiments of the present invention will be described in detail below with reference to the accompanying drawings.

### Embodiment 1

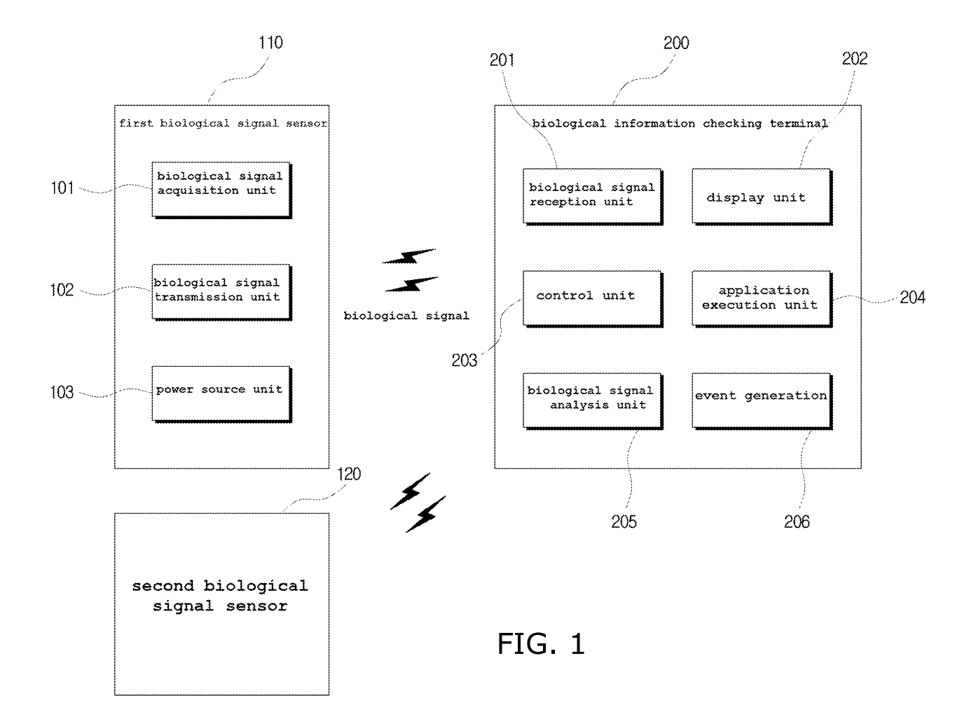

FIG. 1 is a block diagram showing a system for presenting changes in pulses by means of a pulse analysis application according to a preferred first embodiment of the present invention.

The system for presenting changes in pulses by means of a pulse analysis application according to the present embodiment includes: first and second biological signal sensors 110 and 120 configured to acquire biological signals, and to wirelessly transmit the acquired biological signals; and a biological information checking terminal 200 configured to display the biological signals transmitted by the first and second biological signal sensors 110 and 120, to calculate synchronization between changes in the plurality of biological signals by means of a pulse analysis application, and to display the result of the calculation as an event.

In this case, the first biological signal sensor 110 is a biological signal sensor configured to detect a user's own biological signal, and the second biological signal sensor 120 is a biological signal sensor configured to detect the biological signal of another person. Although the pulse signals are described as biological signals in the present embodiment, the present invention is not limited thereto. It may be possible to apply one of various biological signals in the same manner and display a change therein.

Since the configurations and operations of the first and second biological signal sensors 110 and 120 are the same, only one of the biological signal sensors (for example, the first biological signal sensor) will be described below for ease of description.

The first biological signal sensor 110 includes: a biological signal acquisition unit 101 configured to acquire the pulse of a measurement target person in the form of a biological signal; a biological signal transmission unit 102 configured to convert the biological signal acquired by the biological signal acquisition unit 101 into a short-distance wireless signal, and to transmit the short-distance wireless signal by means of a wireless method; and a power source unit 103 configured to supply driving power. In this case, although known various short-distance wireless signals may be used as the short-distance wireless signal, a Bluetooth 4.0 signal is described as being used as the short-distance wireless signal in embodiments described herein.

It is preferred that the first and second biological signal sensors 110 and 120 are implemented in the form of bands, detect pulses as biological signals in the state of being worn on the wrists of measurement target persons, convert the detected biological signals into short-distance wireless signals, and transmit the short-distance wireless signals.

In this case, the biological signal acquired by the first biological signal sensor 110 is referred to as a “first pulse signal,” and the biological signal acquired by the second biological signal sensor 120 is referred to as a “second pulse signal.”

Furthermore, although the biological information checking terminal 200 may be implemented as various information devices, the biological information checking terminal 200 will be described as being implemented as a smartphone in the embodiments described therein. The biological information checking terminal 200 includes: a biological signal reception unit 201 configured to receive biological signals transmitted by the first and second biological signal sensors 110 and 120; a display unit 202 configured to visually display the received biological signals; a control unit 203 configured to control the analysis of a plurality of pieces of received biological information, and to control the occurrence of an event based on the result of the analysis; and an application execution unit 204 configured to execute a pulse analysis application in conjunction with the control unit 203.

In this case, the pulse analysis application refers to software which is adapted to represent two pulse signals by the numbers of lamps, to calculate synchronization between changes in pulses, and to acquire the result of the calculation, or which is adapted to represent two pulse signals by color concentrations, to calculate synchronization between changes in pulses, and to acquire the result of the calculation.

Furthermore, the biological information checking terminal 200 may further include: a biological signal analysis unit 205 configured to analyze biological signals by means of a method of calculating synchronization between changes in first and second pulse signals by means of the pulse analysis application executed by the application execution unit 204; and an event generation unit 206 configured to generate an event corresponding to synchronization between the changes in pulses, analyzed by the biological signal analysis unit 205, under the control of the control unit 203.

In this case, it is preferred that the event generation unit 206 varies a sound effect or automatically selects and outputs corresponding music based on the result of the calculation of synchronization between changes in pulses.

The operation of the system for presenting changes in pulses by means of a pulse analysis application according to the preferred first embodiment of the present invention, which is configured as described above, will be described below more specifically.

First, the first and second biological signal sensors 110 and 120 configured to acquire the pulse signals of measurement targets as biological signals are worn on a user's own wrist and the wrist of another person who is a target for the determination of synchronization, respectively. In this case, the determination of synchronization refers to the determination of synchronization between changes in pulses in a specific state (for example, in an excited state).

The biological signal acquisition unit 101 of the first biological signal sensor 110 acquires the pulse of the measurement target person as a biological signal by using a already known pulse sensor (for example, PR320) for the purpose of measuring a pulse. The pulse signal acquired as described above is converted into a short-distance wireless signal (a Bluetooth signal) and wirelessly transmitted by means of a Bluetooth method via the biological signal transmission unit 102.

Furthermore, the second biological signal sensor 120 operates in the same manner as the first biological signal sensor 110, detects a pulse signal as a biological signal, and wirelessly transmits the biological signal by means of a Bluetooth method.

The pulse signals acquired from the user and another person are received via the biological signal reception unit 201 of the biological information checking terminal 200, i.e., a smartphone. In this case, it is assumed that the pulse analysis application is executed via the application execution unit 204 in the biological information checking terminal 200. It is preferred that the biological signal reception unit 201 acquires the biological signal by means of a Bluetooth communication method. In this case, in the Bluetooth method, a communication connection between devices is established through a procedure called pairing. It is preferred that the biological information checking terminal 200 receives a biological signal through pairing with one biological signal sensor first, releases the connection, and then receives a biological signal through pairing with the other biological signal sensor. In other words, all the pulse signals generated by the two biological signal sensors are received through alternating operation.

The display unit 202 of the biological information checking terminal 200 displays biological information, received by the biological signal reception unit 201, on a screen. This enables the user to determine a change in his or her pulse and a change in the pulse of another person by means of his or her smartphone in a specific state (for example, in an excited state).

Once the biological signals have been received, the control unit 203 controls the biological signal analysis unit 205 so that the biological signal analysis unit 205 calculates synchronization between a change in the pulse of the user and a change in the pulse of the other person.

The biological signal analysis unit 205 calculates synchronization between the changes in the first and second pulse signals by means of a pulse analysis program, included in the pulse analysis application, in response to the control.

In this case, in the calculation of synchronization between changes in pulses, the first and second pulse signals are converted into specific bar graphs, and synchronization between changes in pulses is calculated in a specific state by overlapping the bar graphs on each other. The conversion of the pulse signals into the bar graphs may be performed using a method of preparing a bar graph table in accordance with pulse levels in advance and, when pulse levels are detected, searching the bar graph table and extracting corresponding bar graphs.

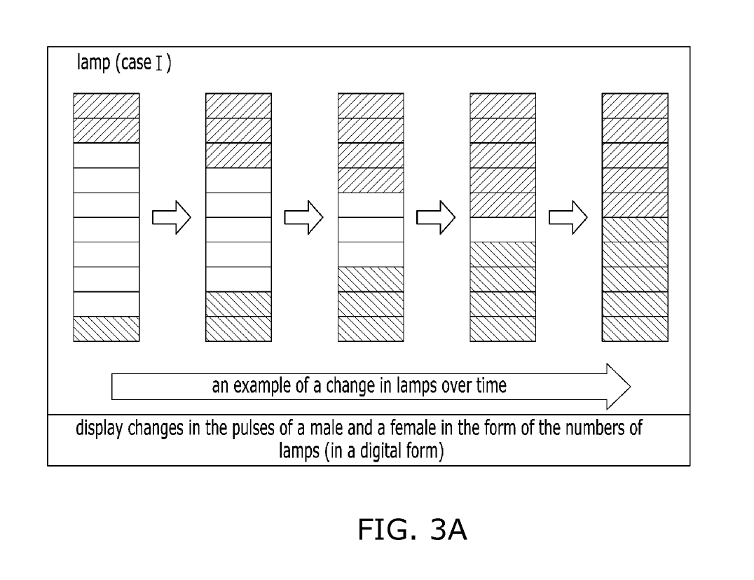

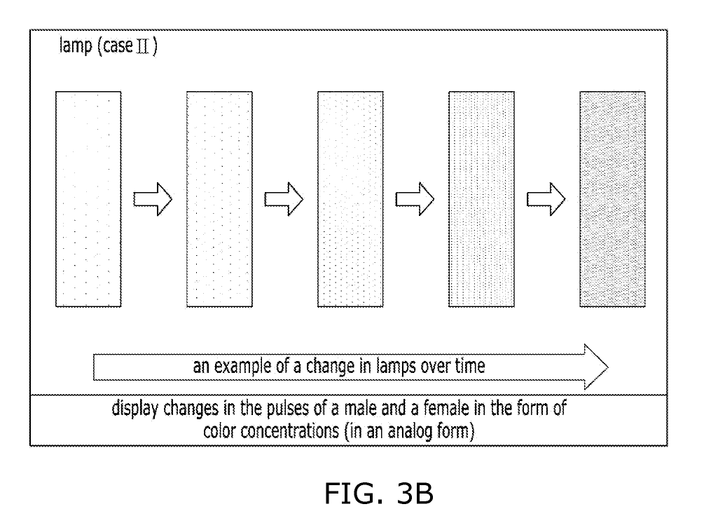

Furthermore, the overlapping of the two bar graphs based on the two pulse signals is performed using a method of locating the bar graph of the first pulse signal in an upper part and the bar graph of the second pulse signal in a lower part and acquiring changes in the two bar graphs based on changes in pulses as a calculation result, as shown in FIG. 3a.

In another method, in the calculation of the synchronization between the changes in pulses, synchronization between changes in pulses in a specific state is calculated by converting the first and second pulse signals into the concentrations of a specific color and overlapping the concentrations of the color on each other. The conversion of the pulse signals into the concentrations of a color may be performed using a method of preparing a color concentration table in accordance with pulse levels and, when pulse levels are detected, searching the color concentration table and extracting corresponding color concentrations.

The overlapping of the two pieces of color concentration data based on the two pulse signals may be performed using a method of overlapping the color concentration data of the first pulse signal and the color concentration data of the second pulse signal on each other, as shown in FIG. 3b. As another method, there may be used a method of converting individual pulse signals into respective pieces of color concentration data and separately displaying the color concentration data.

The result of the calculation of the synchronization between the changes in pulses, which is acquired in the form of bar graphs or color concentrations, is displayed on a screen via the display unit 202, thereby enabling the user to determine his or her own pulse change state and the pulse change state of a counterpart in a specific state and synchronization between a change in his or her pulse and a change in the pulse of the counterpart.

In the same manner, the other person may determine synchronization between his or her own change in pulse and the change in the pulse of the user by means of the same method.

Meanwhile, the result of the calculation of the synchronization between the changes in pulses acquired by the biological signal analysis unit 205 is transferred to the event generation unit 206, and then the event generation unit 206 generates an event corresponding to the transferred result of the calculation of the synchronization between the changes in pulses.

In this case, although the event may be generated in various forms, it is preferred that the event is generated using a function which is basically provided in a common smartphone.

For example, a sound effect (the pitch of sound) may be varied or corresponding music may be automatically selected and output based on the result of the calculation of the synchronization between the changes in pulses. The variation of the pitch of sound or the selection of music based on the synchronization between the changes in pulses may be variably set by a user in advance.

### Embodiment 2

Meanwhile, the above-described embodiment 1 corresponds to a case where an event based on the result of the calculation of synchronization between changes in pulses is generated in a smartphone itself. This embodiment may be extended to a case where an additional event is also generated using another event generation device.

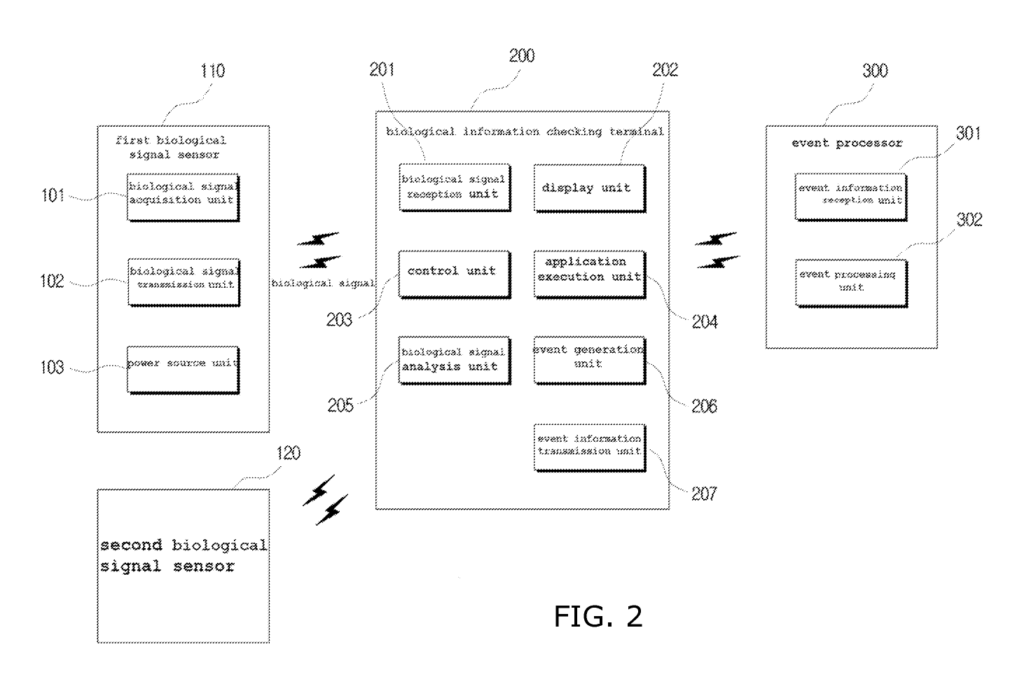

As shown in FIG. 2, a system for presenting changes in pulses by means of a pulse analysis application according to a second embodiment of the present invention includes: first and second biological signal sensors 110 and 120 configured to acquire biological signals, and to wirelessly transmit the acquired biological signals; a biological information checking terminal 200 configured to display the biological signals transmitted by the first and second biological signal sensors 110 and 120, to calculate synchronization between changes in the plurality of biological signals by means of a pulse analysis application, to display the result of the calculation as an event, and to convert the event into a short-distance wireless signal and transmit the short-distance wireless signal; and an event processor 300 configured to process the event signal transmitted by the biological information checking terminal 200, and to generate an event corresponding to the synchronization between the changes in pulses.

In this case, the configurations and operations of the first and second biological signal sensors 110 and 120 are the same as those of the first and second biological signal sensors 110 and 120 shown in FIG. 1.

Furthermore, the biological information checking terminal 200 is formed by adding an event information transmission unit 207 configured to transmit only event information as a short-distance wireless signal to the configuration of the biological information checking terminal shown in FIG. 1.

Furthermore, the event processor 300 includes: an event information reception unit 301 configured to receive event information by means of a short-distance wireless communication signal; and an event processing unit 302 configured to represent synchronization between changes in pulses in accordance with the event information received by the event information reception unit 301.

It is preferred that the event processing unit 302 represents synchronization between changes in pulses by the combination of the numbers of lamps in the form of an event, represents synchronization between changes in pulses by a change in color concentration, or processes an event by emitting a scent corresponding to synchronization between changes in pulses.

The operation of the system for presenting changes in pulses by means of a pulse analysis application according to the preferred second embodiment of the present invention, which is configured as described above, will be described more specifically below.

First, the first and second biological signal sensors 110 and 120 configured to acquire the pulse signals of measurement targets as biological signals are worn on a user's own wrist and the wrist of another person who is a target for the determination of synchronization, respectively. In this case, the determination of synchronization refers to the determination of synchronization between changes in pulses in a specific state (for example, in an excited state).

The biological signal acquisition unit 101 of the first biological signal sensor 110 acquires the pulse of the measurement target person as a biological signal by using a already known pulse sensor (for example, PR320) for the purpose of measuring a pulse. The pulse signal acquired as described above is converted into a short-distance wireless signal (a Bluetooth signal) and wirelessly transmitted by means of a Bluetooth method via the biological signal transmission unit 102.

Furthermore, the second biological signal sensor 120 operates in the same manner as the first biological signal sensor 110, detects a pulse signal as a biological signal, and wirelessly transmits the biological signal by means of a Bluetooth method.

The pulse signals acquired from the user and another person are received via the biological signal reception unit 201 of the biological information checking terminal 200, i.e., a smartphone. In this case, it is assumed that the pulse analysis application is executed via the application execution unit 204 in the biological information checking terminal 200. It is preferred that the biological signal reception unit 201 acquires the biological signal by means of a Bluetooth communication method. In this case, in the Bluetooth method, a communication connection between devices is established through a procedure called pairing. It is preferred that the biological information checking terminal 200 receives a biological signal through pairing with one biological signal sensor first, releases the connection, and then receives a biological signal through pairing with the other biological signal sensor. In other words, all the pulse signals generated by the two biological signal sensors are received through alternating operation.

The display unit 202 of the biological information checking terminal 200 displays biological information, received by the biological signal reception unit 201, on a screen. This enables the user to determine a change in his or her pulse and a change in the pulse of another person by means of his or her smartphone in a specific state (for example, in an excited state).

Once the biological signals have been received, the control unit 203 controls the biological signal analysis unit 205 so that the biological signal analysis unit 205 calculates synchronization between a change in the pulse of the user and a change in the pulse of the other person.

The biological signal analysis unit 205 calculates synchronization between the changes in the first and second pulse signals by means of a pulse analysis program, included in the pulse analysis application, in response to the control.

In this case, in the calculation of synchronization between changes in pulses, the first and second pulse signals are converted into specific bar graphs, and synchronization between changes in pulses is calculated in a specific state by overlapping the bar graphs on each other. The conversion of the pulse signals into the bar graphs may be performed using a method of preparing a bar graph table in accordance with pulse levels in advance and, when pulse levels are detected, searching the bar graph table and extracting corresponding bar graphs.

Furthermore, the overlapping of the two bar graphs based on the two pulse signals is performed using a method of locating the bar graph of the first pulse signal in an upper part and the bar graph of the second pulse signal in a lower part and acquiring changes in the two bar graphs based on changes in pulses as a calculation result, as shown in FIG. 3a.

In another method, in the calculation of the synchronization between the changes in pulses, synchronization between changes in pulses in a specific state is calculated by converting the first and second pulse signals into the concentrations of a specific color and overlapping the concentrations of the color on each other. The conversion of the pulse signals into the concentrations of a color may be performed using a method of preparing a color concentration table in accordance with pulse levels and, when pulse levels are detected, searching the color concentration table and extracting corresponding color concentrations.

The overlapping of the two pieces of color concentration data based on the two pulse signals may be performed using a method of overlapping the color concentration data of the first pulse signal and the color concentration data of the second pulse signal on each other, as shown in FIG. 3b. As another method, there may be used a method of converting individual pulse signals into respective pieces of color concentration data and separately displaying the color concentration data.

The result of the calculation of the synchronization between the changes in pulses, which is acquired in the form of bar graphs or color concentrations, is displayed on a screen via the display unit 202, thereby enabling the user to determine his or her own pulse change state and the pulse change state of a counterpart in a specific state and synchronization between a change in his or her pulse and a change in the pulse of the counterpart.

In the same manner, the other person may determine synchronization between his or her own change in pulse and the change in the pulse of the user by means of the same method.

Meanwhile, the result of the calculation of the synchronization between the changes in pulses acquired by the biological signal analysis unit 205 is transferred to the event generation unit 206, and then the event generation unit 206 generates an event corresponding to the transferred result of the calculation of the synchronization between the changes in pulses.

In this case, although the event may be generated in various forms, it is preferred that the event is generated using a function which is basically provided in a common smartphone.

For example, a sound effect (the pitch of sound) may be varied or corresponding music may be automatically selected and output based on the result of the calculation of the synchronization between the changes in pulses. The variation of the pitch of sound or the selection of music based on the synchronization between the changes in pulses may be variably set by a user in advance.

Furthermore, the event information transmission unit 207 converts the result of the calculation of the synchronization between the changes in pulses, acquired by the biological signal analysis unit 205, into a short-distance wireless signal, and transmits the short-distance wireless signal to the event processor 300.

The event processor 300 receives the result of the calculation of the synchronization between the changes in pulses as event information via the event information reception unit 301, and the event processing unit 302 processes an event based on the result of the calculation of the synchronization between the changes in pulses by emitting a scent through the driving of a scent generation device based on the result of the calculation of the synchronization between the changes in pulses.

Furthermore, the event processing unit 302 processes an event by representing the result of the calculation of synchronization between changes in pulses in the form of bar graphs in the same manner as the biological information checking terminal 200. Alternatively, the event processing unit 302 may represent the result of the calculation of synchronization between changes in pulses by color concentrations.

As described above, the present invention can display the pulse change state of a user and the pulse change state of another person in a specific state via a smartphone pulse application, and can calculate synchronization between a change in the pulse of a user and a change in the pulse of another person, thereby enabling the result of the calculation to be mutually checked visually, acoustically, and olfactorily over time or according to a change in an excited state.

The present invention is applied to technology which calculates synchronization between changes in pulses through the overlapping of the pulses of a user and another person and presents the result of the calculation.

According to the present invention, there is provided the system for presenting changes in pulses by means of a pulse analysis application, which detects a user's own pulse and the pulse of another person, calculates synchronization between changes in the user's own pulse and the pulse of the other person by means of a pulse analysis application of a smartphone, and presents the result of the calculation, thereby enabling the climax of a mutual change in pulses to be determined.

Although the present invention has been described based on the embodiments specifically, the present invention is not limited to the embodiments. Furthermore, it will be apparent to a person having ordinary knowledge in the art that various modifications and alterations can be made to the present invention without departing from the gist of the present invention.

What is claimed is:

1. A system for presenting changes in pulses by means of a pulse analysis application, the system displaying synchronization between changes in pulses by means of a pulse analysis application of a smartphone, the system comprising:
a plurality of biological signal sensors configured to acquire biological signals, and to wirelessly transmit the acquired biological signals; and
a biological information checking terminal configured to display the biological signals transmitted by the plurality of biological signal sensors, to calculate synchronization between the plurality of biological signals by means of a pulse analysis application, and to display a result of the calculation as an event;
wherein the biological information checking terminal comprises: a control unit configured to control analysis of a plurality of pieces of received biological information and to control an occurrence of an event based on a result of the analysis; an application execution unit configured to execute the pulse analysis application in conjunction with the control unit; and a biological signal analysis unit configured to analyze the biological signals by means of a method of calculating synchronization between changes in first and second pulse signals by means of the pulse analysis application executed by the application execution unit; and
wherein the biological signal analysis unit, by means of a pulse analysis program included in the pulse analysis application, analyzes the biological signals by converting the first and second pulse signals into respective specific bar graphs and overlapping the bar graphs on each other, thereby calculating synchronization between changes in pulses in a specific state.

2. The system of claim 1, wherein the plurality of biological signal sensors are worn on wrists of biological signal measurement target persons in band forms, detect pulses as biological signals, convert the detected biological signals into short-distance wireless signals, and transmit the short-distance wireless signals.

3. The system of claim 1, wherein the biological information checking terminal is implemented as a smartphone, and the biological information checking terminal further comprises a display unit configured to visually display the received biological signals.

4. The system of claim 1, wherein the biological information checking terminal further comprises an event generation unit configured to generate an event corresponding to the synchronization between the changes in pulses analyzed by the biological signal analysis unit under a control of the control unit.

5. The system of claim 4, wherein:
the biological information checking terminal visually or acoustically represents a result of analysis of the biological information acquired through calculation of the synchronization between the changes in pulses; and
to the event generation unit varies a sound effect or automatically selects and outputs corresponding music based on a result of the calculation of the synchronization between the changes in pulses.

6. The system of claim 1, wherein the biological information checking terminal represents the synchronization between the changes in pulses by a combination of numbers of lamps as an event or by a change in color concentration.

7. A system for presenting changes in pulses by means of a pulse analysis application, the system calculating synchronization between changes in pulses by means of a pulse analysis application of a smartphone and presenting a result of the calculation of the synchronization between the changes in pulses, the system comprising:
a plurality of biological signal sensors configured to acquire biological signals, and to wirelessly transmit the acquired biological signals; and
a biological information checking terminal configured to display the biological signals transmitted by the plurality of biological signal sensors, to calculate synchronization between the plurality of biological signals by means of a pulse analysis application, to display a result of the calculation as an event, to convert the event into a short-distance wireless signal, and to transmit the short-distance wireless signal; and
an event processor configured to generate an event corresponding to the synchronization between the changes in pulses by processing the event signal transmitted by the biological information checking terminal;
wherein the biological information checking terminal comprises: a control unit configured to control analysis of a plurality of pieces of received biological information and to control an occurrence of an event based on a result of the analysis; an application execution unit configured to execute the pulse analysis application in conjunction with the control unit; and a biological signal analysis unit configured to analyze the biological signals by means of a method of calculating synchronization between changes in first and second pulse signals by means of the pulse analysis application executed by the application execution unit; and
wherein the biological signal analysis unit, by means of a pulse analysis program included in the pulse analysis application, analyzes the biological signals by converting the first and second pulse signals into respective specific bar graphs and overlapping the bar graphs on each other, thereby calculating synchronization between changes in pulses in a specific state.

8. The system of claim 7, wherein the biological information checking terminal further comprises a display unit configured to visually display the received biological signals.

9. The system of claim 7, wherein the biological information checking terminal further comprises:
an event generation unit configured to generate an event corresponding to the synchronization between the changes in pulses analyzed by the biological signal analysis unit under a control of the control unit; and
an event information transmission unit configured to transmit event information corresponding to the synchronization between the changes in pulses analyzed by the biological signal analysis unit under a control of the control unit.

10. The system of claim 7, wherein:
the event processor comprises an event processing unit configured to represent the synchronization between the changes in pulses in accordance with received event information; and
the event processing unit represents the synchronization between the changes in pulses by a combination of numbers of lamps as an event or by a change in color concentration.

11. The system of claim 10, wherein the event processing unit processes the event by emitting a scent corresponding to the synchronization between the changes in pulses.
